(19) United States
(12) Patent Application Publication
(10) Pub. No.: US 2016/0256632 A1
(43) Pub. Date: Sep. 8, 2016

(54) FLUID TRANSFER DEVICES AND METHODS OF USE

(71) Applicant: ICU Medical, Inc., San Clemente, CA (US)

(72) Inventor: Thomas F. Fangrow, Mission Viejo, CA (US)

(21) Appl. No.: 15/156,257

(22) Filed: May 16, 2016

Related U.S. Application Data
(63) Continuation of application No. PCT/US2014/066645, filed on Nov. 20, 2014.
(60) Provisional application No. 61/907,995, filed on Nov. 22, 2013.

(51) Int. Cl. A61M 5/31; A61J 1/20

(52) U.S. Cl. CPC A61M 5/31; A61J 1/2096; A61J 1/2048; A61J 1/2072; A61M 2005/3123

(57) ABSTRACT

Embodiments disclosed herein relate to systems and methods for transferring fluids. A fluid pathway can extend between a first fluid container and a second fluid container. An air chamber can be in fluid communication with the fluid pathway between the first fluid container and the second fluid container. During normal operating pressures, air can be maintained in the air chamber. During low pressure conditions (e.g., caused by a malfunction), the air in the air chamber can expand to a sensing location (e.g., in the fluid pathway). An air sensor can detect the presence of the air at the sensing location, and can provide an indication of a possible low pressure condition.

### RELATED APPLICATIONS

This application claims the benefit under 35 U.S.C. §120 and 35 U.S.C. §365(c) as a continuation of International PCT Patent Application No. PCT/US2014/066645, designating the United States, with an international filing date of Nov. 20, 2014, and titled FLUID TRANSFER DEVICES AND METHODS OF USE, which claims the benefit under 35 U.S.C. §119(e) of U.S. Provisional Patent Application No. 61/907,995, filed Nov. 22, 2013, and titled FLUID TRANSFER DEVICES AND METHODS OF USE. The entirety of each of the above-identified applications is hereby incorporated by reference herein and made a part of this disclosure.

### INCORPORATION BY REFERENCE

U.S. Pat. No. 8,522,832 (the “'832 patent”), titled “FLUID TRANSFER DEVICES AND METHODS OF USE,” filed on Jul. 28, 2010 as U.S. patent application Ser. No. 12/845,548, and granted on Sep. 3, 2013, is hereby incorporated by reference in its entirety and made part of this specification for all that it discloses.

International PCT Patent Publication No. WO 2013/096911 (the “'911 Publication”), titled “FLUID TRANSFER DEVICES AND METHODS OF USE,” filed on Dec. 21, 2012 as International PCT Patent Application No. PCT/US2012/071493, and published on Jun. 27, 2013, is hereby incorporated by reference in its entirety and made part of this specification for all that it discloses.

Any component, structure, material, step, method, or system illustrated and/or described in either of the '832 patent or the '911 Publication can be used with or instead of any component, structure, material, step, method, or system illustrated and/or described in this specification.

### BACKGROUND

1. Field of the Disclosure

Some embodiments of this disclosure relate to devices and methods for transferring fluids, and more particularly to devices and methods for transferring medical fluids from a first fluid container to a second fluid container.

2. Description of the Related Art

In some circumstances, it can be desirable to transfer one or more fluids between containers. In the medical field, it can be desirable to dispense medical (e.g., medication) fluids in precise amounts. In some cases, it can be desirable to dispense potentially dangerous fluids (e.g., chemotherapy or immunosuppressive drugs). Some fluid dispensing systems suffer from various drawbacks, including high cost, low efficiency, intensive labor demands, and excessive fluid or vapor leakage. Some fluid dispensing systems can have insufficient precision, for example, due to the transfer of some air along with the fluid. Some automated fluid dispensing systems can be susceptible to failure without the ability to detect the failure or alert an operator. Some embodiments disclosed herein overcome one or more of these disadvantages.

### SUMMARY OF CERTAIN EMBODIMENTS

For purposes of summarizing the disclosure, certain aspects, advantages and novel features have been described herein. It is to be understood that not necessarily all such advantages can be achieved in accordance with any particular embodiment disclosed herein. Thus, the features disclosed herein can be embodied or carried out in a manner that achieves or optimizes one advantage or group of advantages as taught herein without necessarily achieving other advantages as can be taught or suggested herein.

Various embodiments disclosed herein can relate to A method of transferring fluid from a fluid source container to a syringe. The method can include retracting a plunger on a syringe to draw a first volume of fluid from a fluid source container, through a source fluid pathway, and into the syringe. An air chamber can be in fluid communication with the source fluid pathway between the fluid source container and the syringe. The method can include retracting the plunger on the syringe to draw a second volume of fluid from the source fluid pathway into the syringe, and fluid can be impeded from exiting the source container such that air in the air chamber expands to a sensing location in the fluid pathway between the source container and the syringe. The method can include identifying the air at the sensing location in the source fluid pathway between the source container and the syringe with an air sensor and in response to identifying the air at the sensing location in the fluid pathway, stopping the retracting of the plunger on the syringe.

The method can include in response to identifying the air at the sensing location in the fluid pathway, providing an indication that a malfunction may have occurred. The method can include in response to identifying the air at the sensing location in the fluid pathway, providing an indication that the fluid source container may be empty.

The first volume of fluid can pass through a source check valve in the source fluid pathway between the fluid source container and the syringe, and the source check valve can be configured to impede fluid from passing through the source check valve towards the fluid source container.

The method can include advancing the plunger on the syringe to drive fluid from the syringe, through a destination fluid pathway, and towards a fluid destination container. The fluid can pass through a destination check valve in the fluid destination pathway between the syringe and the fluid destination container, and the destination check valve is configured to impede fluid from passing through the destination check valve towards the syringe.

Various embodiments disclosed herein can relate to a fluid transfer module that can be configured to be removably attachable to an electronically controlled fluid dispensing system. The fluid transfer module can include a source interface configured to be connected to a fluid source container to provide fluid communication between the source interface and the fluid source container, a destination interface configured to be connected to a fluid destination container to provide fluid communication between the destination interface the fluid destination container, and an intermediate container, or an intermediate interface configured to be connected to an intermediate container. A source fluid pathway can extend between the source interface and the intermediate container, or the intermediate interface, and the source fluid pathway can be configured to allow passage of fluid from the fluid source container to the intermediate container. The fluid transfer module can include a destination fluid pathway that can extend between the intermediate container, or the intermediate interface, and the destination interface. The destination fluid pathway can be configured to allow passage of fluid from the intermediate container to the fluid destination container. The fluid transfer module can include an air chamber in fluid communication with the source fluid pathway. The air chamber can be configured such that air from the air chamber expands to a sensing location if pressure within the source fluid pathway is below a threshold value. The fluid transfer module can include an interaction portion configured to permit the electronically controlled fluid dispensing system to detect the air that expands from the air chamber to the sensing location. In some embodiments, the sensing location can be in the source fluid pathway.

The fluid transfer module can include a source check valve in the source fluid pathway, and the source check valve can be configured to impede fluid from passing through the source check valve towards the source interface. The air chamber can be positioned between the source interface and the source check valve. The fluid transfer module can include a destination check valve in the destination fluid pathway, and the destination check valve can be configured to impede fluid from passing through the destination check valve towards the intermediate interface or the intermediate container. In some embodiments, the source check valve and the destination check valve can be integrally formed as a single check valve assembly.

The fluid transfer module can include a main body, and the main body can include a source attachment portion configured to couple the source interface to the main body. The fluid transfer module can include an air chamber module that include the air chamber, a main body attachment portion that is configured to couple the air chamber module to the source attachment portion of the main body, and a source attachment portion configured to couple the air chamber module to the source interface.

In some embodiments, an opening can couple the air chamber to the source fluid pathway. The air chamber can be disposed above the opening.

The source interface can include an aperture and a valve, and the valve can be configured to close the aperture when the fluid source container is decoupled from the source interface, and the valve can be configured to open the aperture when the fluid source container is coupled to the source interface. The destination interface can include an aperture and a valve, and valve can be configured to close the aperture when the fluid destination container is decoupled from the destination interface, and the valve can be configured to open the aperture when the fluid destination container is coupled to the destination interface.

In some embodiments, at least a portion of the source fluid pathway can overlap at least a portion of the destination fluid pathway.

The interaction portion can include a substantially transparent portion of the fluid transfer module.

The fluid transfer module can be configured such that air enters a fluid source container as fluid is withdrawn from the fluid source container. The fluid transfer module can include a fluid source container and an adapter disposed between the source interface and fluid source container. The adapter can include an air inlet and a barrier configured such that air enters the fluid source container as fluid is withdrawn from the fluid source container.

Various embodiments disclosed herein can relate to a fluid transfer system that can include an electronically controlled fluid dispensing system and a fluid transfer module removably attached to the electronically controlled fluid dispensing system. The electronically controlled fluid dispensing system can include an air sensor configured to detect air at the sensing location. The air sensor can include an optical sensor. The electronically controlled fluid dispensing system can include an actuator configured to transfer fluid from the fluid source container to the intermediate container, and/or to transfer fluid from the intermediate container to the fluid destination container. The intermediate container can include a syringe having a plunger. The actuator can be coupled to the plunger, and the electronically controlled fluid dispensing system can include a motor configured to move the actuator to retract and advance the plunger of the syringe.

Various embodiments disclosed here in can relate to a fluid transfer module, which can include a first interface configured to be connected to a first fluid container, a second container, or a second interface configured to be connected to a second fluid container, a first fluid pathway extending between the first interface and the second fluid container, or the second interface, and an air chamber in fluid communication with the first fluid pathway.

The fluid transfer module can include a third interface configured to be connected to a third fluid container and a second fluid pathway extending between the second fluid container, or the second interface, and the third interface.

The fluid transfer module can include a first check valve in the first fluid pathway, and the first check valve can be configured to impede fluid from passing through the first check valve towards the first interface. The air chamber can be positioned between the first interface and the first check valve. The fluid transfer module can include a second check valve in the second fluid pathway, and the second check valve can be configured to impede fluid from passing through the second check valve towards the second interface or the second container. The first check valve and the second check valve can be integrally formed as a single check valve assembly.

The air chamber can be configured such that air from the air chamber can expand to a sensing location in response to reduced pressure in the first fluid pathway. The sensing location can be in the first fluid pathway.

The fluid transfer module can include a main body, and the air chamber can be positioned between the main body and the first interface. The fluid transfer module can include an air chamber module that has the air chamber, a first attachment portion configured to couple the air chamber module to the first interface, and a main body attachment portion that is configured to couple the air chamber module to a first attachment portion of the main body. An opening can couple the air chamber to the first fluid pathway. The air chamber can be disposed above the opening.

The first interface can include an aperture and a valve, and the valve can be configured to close the aperture when the first fluid container is decoupled from the first interface, and the valve can be configured to open the aperture when the first fluid container is coupled to the first interface. The fluid transfer module can include a third interface that has an aperture and a valve, and the valve can be configured to close the aperture when a third fluid container is decoupled from the third interface, and the valve can be configured to open the aperture when the third fluid container is coupled to the third interface.

The fluid transfer module can include an interaction portion that is configured to permit an air sensor to detect air that expands from the air chamber to a sensing location. The interaction portion can include a substantially transparent portion of the fluid transfer module.

In some embodiments, air can enter the first container as fluid is withdrawn from the first container. The fluid transfer module can include a first fluid container and an adapter disposed between the first interface and first fluid container. The adapter can include an air inlet and a barrier configured such that air enters the first fluid container as fluid is withdrawn from the first fluid container.

Various embodiments disclosed herein can relate to a fluid transfer system that can include an electronically controlled fluid dispensing system and a fluid transfer module removably attached to the electronically controlled fluid dispensing system. The electronically controlled fluid dispensing system can include an air sensor configured to detect expanded air from the air chamber. The air sensor can include an optical sensor. The electronically controlled fluid dispensing system can include an actuator configured to transfer fluid from the first fluid container to the second container. The second container can include a syringe having a plunger. The actuator can be coupled to the plunger, and the electronically controlled fluid dispensing system can include a motor configured to move the actuator to retract and advance the plunger of the syringe.

Various embodiments disclosed herein can relate to a fluid transfer module that can include a first fluid container, a second fluid container, a first fluid pathway extending between the first fluid container and the second fluid container, an air chamber in fluid communication with the first fluid pathway.

The fluid transfer module can include a third fluid container and a second fluid pathway extending between the second fluid container and the third fluid container.

The fluid transfer module can include a first check valve in the first fluid pathway, and the first check valve can be configured to impede fluid from passing through the first check valve towards the first fluid container. The air chamber can be positioned between the first fluid container and the first check valve. The fluid transfer module can include a second check valve in the second fluid pathway, and the second check valve can be configured to impede fluid from passing through the second check valve towards the second container. In some embodiments, the first check valve and the second check valve can be integrally formed as a single check valve assembly.

The air chamber can be configured such that air from the air chamber can expand to a sensing location in response to reduced pressure in the first fluid pathway. The sensing location can be in the first fluid pathway.

The fluid transfer module can include a main body, and the air chamber can be positioned between the main body and the first fluid container. The fluid transfer module can include the air chamber, a first attachment portion configured to couple the air chamber module to the first fluid container, and a main body attachment portion that is configured to couple the air chamber module to a first attachment portion of the main body. An opening can couple the air chamber to the first fluid pathway. The air chamber can be disposed above the opening.

A first interface can couple the first fluid container to a main body, and the first interface can include an aperture and a valve. The valve can be configured to close the aperture when the first fluid container is decoupled from the first interface, and the valve can be configured to open the aperture when the first fluid container is coupled to the first interface. The fluid transfer module can include a third interface that couples the third container to a main body, and the third interface can include an aperture and a valve. The valve can be configured to close the aperture when a third fluid container is decoupled from the third interface, and the valve can be configured to open the aperture when the third fluid container is coupled to the third interface.

The fluid transfer module can include an interaction portion that is configured to permit an air sensor to detect air that expands from the air chamber to a sensing location. The interaction portion can include a substantially transparent portion of the fluid transfer module.

In some embodiments, air can enter the first container as fluid is withdrawn from the first fluid container. The fluid transfer module can include an adapter configured to couple the first fluid container to the first fluid pathway, and the adapter can include an air inlet and a barrier configured such that air enters the first fluid container as fluid is withdrawn from the first fluid container.

Various embodiments disclosed herein can relate to a fluid transfer system that can include an electronically controlled fluid dispensing system and a fluid transfer module removably attached to the electronically controlled fluid dispensing system. The electronically controlled fluid dispensing system can include an air sensor configured to detect expanded air from the air chamber. The air sensor comprises an optical sensor. The electronically controlled fluid dispensing system can include an actuator configured to transfer fluid from the first fluid container to the second container. The second container can include a syringe having a plunger. The actuator can be coupled to the plunger, and the electronically controlled fluid dispensing system can include a motor configured to move the actuator to retract and advance the plunger of the syringe.

### BRIEF DESCRIPTION OF THE DRAWINGS

FIG. 1 schematically shows an example embodiment of a fluid transfer system.

FIG. 2 schematically shows another example embodiment of a fluid transfer system.

FIG. 3 is a perspective, cross-sectional view of an example embodiment of a fluid transfer module.

FIG. 4 is an exploded, perspective, cross-sectional view of the fluid transfer module of FIG. 3.

FIG. 5 is a perspective, cross-sectional view of an example embodiment of a main body for a fluid transfer module.

FIG. 6 is an exploded, perspective, cross-sectional view of the main body of FIG. 5.

FIG. 7 is a perspective, cross-sectional view of an example embodiment of an air chamber module.

FIG. 8 is an exploded, perspective, cross-sectional view of the air chamber module of FIG. 7.

FIG. 9 is a cross-sectional view of an example embodiment of a fluid transfer module with fluid in the fluid pathways.

FIG. 10 is a cross-sectional view of the fluid transfer module of FIG. 9 with reduced pressure therein.

FIG. 11 is a cross-sectional view showing examples of various possible sensing locations on a fluid transfer module.

FIG. 12 is a cross-sectional view of an example embodiment of a fluid source container and adapter.

FIG. 13 shows a side view of an example embodiment of a check valve in a closed position.

FIG. 14 shows the check valve of FIG. 13 in the open position.

FIG. 15 shows a side view of another example embodiment of a check valve in the closed position.

FIG. 16 is a side view of the check valve of FIG. 15 in the open position.

FIG. 17 shows a side view of another example embodiment of a check valve in the closed position.

FIG. 18 is a side view of the check valve of FIG. 17 in the open position.

FIG. 19 is a side view of another example embodiment of a check valve in the closed position.

FIG. 20 is a side view of the check valve of FIG. 20 in the open position.

FIG. 21 is a cross-sectional view of an example embodiment of a fluid transfer module having at least one check valve.

### DETAILED DESCRIPTION OF CERTAIN EMBODIMENTS

The following detailed description is now directed to certain specific example embodiments of the disclosure. In this description, reference is made to the drawings wherein like parts are designated with like reference numerals throughout the description and the drawings. The drawings and the description are to be regarded as illustrative examples and not restrictive. It is contemplated that the disclosed example embodiments can be modified in many ways, including that any of the various individual features illustrated and/or described herein can be combined to form various combinations and subcombinations.

In a fluid transfer system, a malfunction-detection system can detect a malfunction that may otherwise impede fluid from transferring properly, to avoid the transfer of an incorrect amount of fluid (e.g., medicinal fluids such as chemotherapy or immunosuppressive drugs), and/or to avoid leakage of harmful fluid or vapors.

In some cases, a pump (e.g., a syringe pump, a peristaltic pump, etc.) can be used to move fluid from a first container to a second container (e.g., the syringe reservoir of the syringe pump or another reservoir). A malfunction-detection system can be configured to detect a malfunction that may impede the fluid from exiting the first container. For example, in some embodiments, an air inlet can be configured to allow air to enter the first container as fluid exits the first container. If air somehow is not able to enter the first container to occupy the space of the fluid that exits the first container, a reduced pressure (e.g., a partial vacuum) can occur in the first container. In some embodiments, the air inlet can include a barrier that is permeable to air and impermeable to fluid. If the barrier becomes wet (e.g., due to excessive shaking or some other improper use of the device), the barrier can have reduced air permeability or it can become impermeable to air. The reduced pressure or partial vacuum formed inside the first container caused by the blockage of air from entering the first container may impede additional fluid from exiting the first container. In some cases, the reduced pressure or partial vacuum could be transferred to the second container, which can be in fluid communication with the first container. A malfunction-detection system can address other malfunctions.

If the system were unable to detect the malfunction that impedes the fluid from exiting the first container, the pump could continue to try to move fluid into the second container (e.g., by continuing to retract the plunger of the syringe), which could cause the pressure inside the second container to drop. As the pressure drops, small amounts of air in the second container could expand to occupy a significant volume in the second container. The system could transfer the expanded volume of air as though it were the fluid that is intended to be transferred, which could result in a lower than desired transfer of fluid.

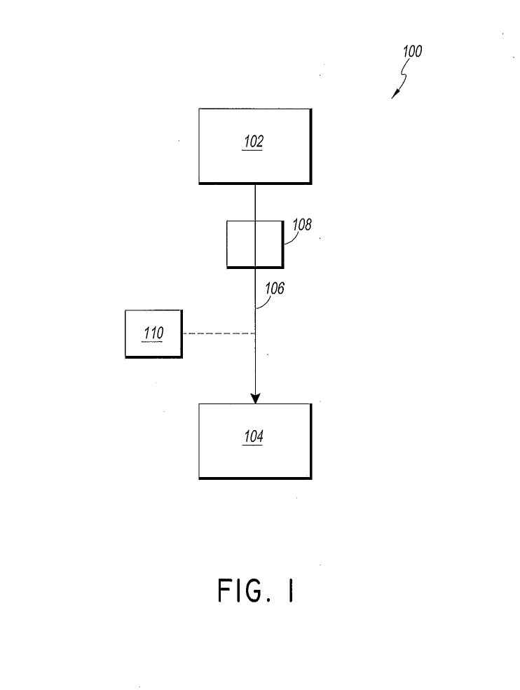

FIG. 1 schematically shows an example embodiment of a fluid transfer system 100. The fluid transfer system 100 can be configured to detect a malfunction that impedes the proper transfer of fluid. The fluid transfer system 100 can include features similar to, or the same as, those discussed above, or those discussed in connection with various other embodiments disclosed herein, including those incorporated by reference in this specification. The fluid transfer system 100 can include any features illustrated or described in the '832 patent and/or the '911 Publication. The fluid transfer system 100 can include a first fluid container 102 (e.g., a medication vial), a second fluid container 104 (e.g., a syringe), and a fluid pathway 106 between the first fluid container 102 and the second fluid container 104. An air chamber 108 can be in fluid communication with the fluid pathway 106 that extends between the first container 102 and the second container 104. The air chamber can be sealed off or isolated from ambient air. The system 100 can include an air sensor 110. As used herein, the term “air” is intended to have its broad ordinary meaning, and can include, for example, various combinations of gases that can be identified by the air sensor 110 as being different from the fluid being transferred through the fluid transfer system 100. In some circumstances, the air in the air chamber may include vaporized particles of the fluid being transferred through the fluid transfer system 100. The air sensor 110 can be configured to distinguish between the fluid being transferred through the fluid transfer system 100 and air. For example, the air sensor 110 can be an optical sensor (e.g., including a light source and a light detector). In some embodiments, the air sensor 110 can detect the presence or absence of the fluid (e.g., by absorption of the light passing through the fluid or not passing through the fluid). A detection of an absence of fluid can be the same as a detection of air. For example, if the light detector of the air sensor 110 detects an amount of light from the light source below a threshold level (e.g., due to absorption of the light by the fluid), the air sensor 110 can indicate a detection of fluid. If the light detector of the air sensor 110 detects an amount of light from the light source above a threshold level (e.g., because the fluid was not present to absorb the light), the air sensor 110 can indicate a detection or air. The system 100 can be configured such that the air sensor does not detect the air in the air chamber 108 during normal operation. In the event of a malfunction that causes a reduced pressure in the fluid pathway, the air in the air chamber 108 can expand as a result of the reduced pressure. The air sensor 110 can be positioned to detect the expanded air. The air sensor 110 can provide an indication of possible malfunction in response to the detection of air.

In some embodiments, the air sensor 110 can be used to detect air as an indication that the first container 102 is empty as well as an indication of a possible malfunction. The air sensor 110 can be positioned to detect air in the fluid pathway 106. When the first container 102 becomes empty, air can exit the first container and travel through the fluid pathway 106 towards the second container 104. When the air reaches the sensing location of the air sensor 110, the air sensor 110 can detect the air. In this situation, the detection of air by the air sensor 110 indicates that the first container 102 has become empty. When a malfunction occurs that results in reduced pressure, the air inside the air chamber can expand into the fluid pathway 106. When the expanded air reaches the sensing location of the air sensor 110, the air sensor 110 can detect the air. In this situation the detection of air by the air sensor 110 indicates a reduced pressure inside the fluid pathway 106, which can indicate that a malfunction has occurred.

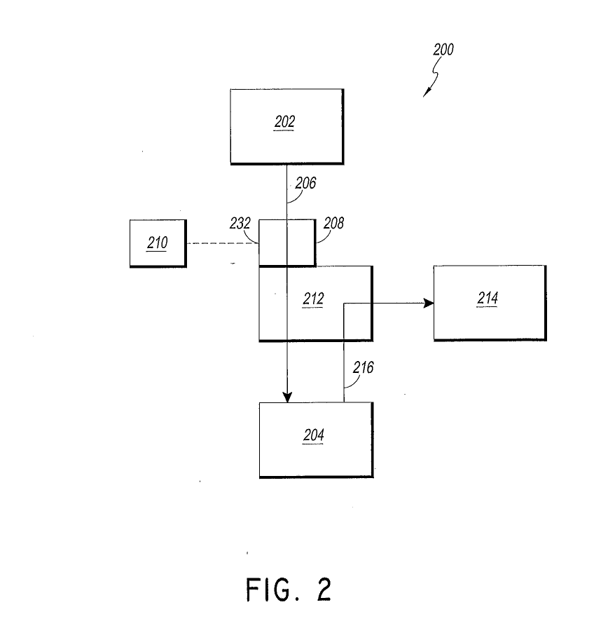

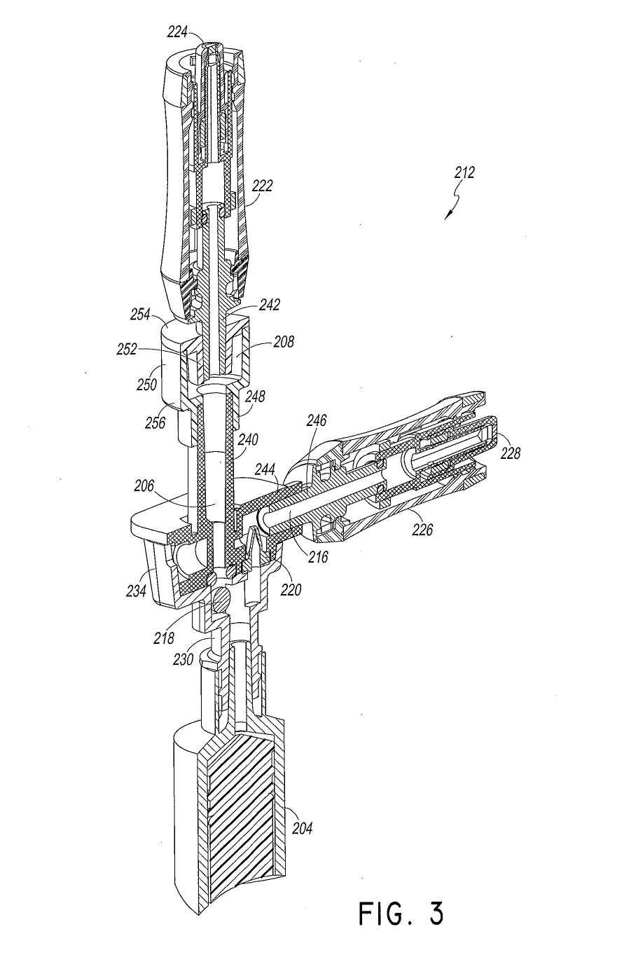

FIG. 2 schematically shows an example embodiment of a fluid transfer system 200. The fluid transfer system 200 can include any feature of the fluid transfer system 100 or various other embodiments disclosed herein. The fluid transfer system 200 can include any feature illustrated and/or described in the '832 patent and/or the '911 Publication. The fluid transfer system 200 can include a fluid source container 202 (e.g., a medication vial), an intermediate container 204 (e.g., a syringe), and a fluid destination container 214 (e.g., an IV bag). A fluid transfer module 212 can be used to couple the fluid source container 202, intermediate container 204, and fluid destination container 214. FIG. 3 is a perspective, cross-sectional view of an example embodiment of a fluid transfer module 212. FIG. 4 is an exploded, perspective, cross-sectional view of the example embodiment of a fluid transfer module 212 of FIG. 3. The fluid transfer module 212 can include any feature illustrated and/or described in the '832 patent and/or the '911 Publication. Many variations are possible. For example, as shown schematically in FIG. 1, in some embodiments, fluid can be transferred from a source container to a destination container without the use of an intermediate container (e.g., without a syringe pump), and in some instances fluid can be transferred using a peristaltic pump or other pump device that does not include an intermediate container (e.g., syringe).

A source fluid pathway 206 can extend between the fluid source container 202 and the intermediate container 204, and can extend through the fluid transfer module 212. A destination fluid pathway 216 can extend between the intermediate container 204 and the fluid destination container 214, and can extend through the fluid transfer module 212. In some embodiments, at least a portion of the source fluid pathway 206 and the destination fluid pathway 216 can overlap. For example, the intermediate container 204 can be a syringe that includes an opening through which both the source fluid pathway 206 and the destination fluid pathway 216 can pass. A source check valve 218 can be positioned in the source fluid pathway 206, which can be configured to permit fluid to pass from the fluid source container 202 to the intermediate container 204 and to impede or prevent the passage of fluid from the intermediate container 204 to the fluid source container 202. A destination check valve 220 can be positioned in the destination fluid pathway 216, which can be configured to permit fluid to pass from the intermediate container 204 to the fluid destination container 214 and to impede or prevent the passage of fluid from the fluid destination container 214 to the intermediate container 204.

The fluid transfer module 212 can include a source interface 222 that is configured to couple the fluid source container 202 to the transfer module 212. The source interface 222 can removably attach to the fluid source container 202 (e.g., via an adapter that is not shown in FIGS. 3 and 4). The source interface 222 can include a valve 224 that is configured to close an aperture of the source interface 222 when the fluid source container 202 is detached from the source interface 222 of the fluid transfer module 212. The valve 224 of the source interface 222 can be configured to open when the fluid source container 202 is attached to the source interface 222 of the fluid transfer module 212. The fluid transfer module 212 can include a destination interface 226 that is configured to couple the fluid destination container 214 to the transfer module 212. The destination interface 226 can removably attach to the fluid destination container 214. The destination interface 226 can include a valve 228 that is configured to close an aperture of the destination interface 226 when the fluid destination container 214 is detached from the destination interface 226 of the fluid transfer module 212. The valve 228 of the destination interface 226 can be configured to open when the fluid destination container 214 is attached to the destination interface 226 of the fluid transfer module 212. In some embodiments, the fluid transfer module 212 can include an intermediate interface 230 that is configured to couple the intermediate container 204 to the transfer module 212. The intermediate interface 230 can removably attach to the intermediate container 204. In some embodiments, the fluid transfer module 212 can include the intermediate container 204 as part of the fluid transfer module 204, and the intermediate interface 230 can be a permanent or temporary attachment, or it can be omitted. Also, in some embodiments, the fluid source container 202 and/or the fluid destination container 214 can be included as part of the fluid transfer module. Thus, in some embodiments, the source interface 222 and/or the destination interface 226 can be a permanent or temporary attachment, or it can be omitted.

The fluid transfer system 200 can include an air chamber 208 and an air sensor 210, which can perform or include any function described and/or illustrated in connection with the air chamber 108 and the air sensor 110 or various other embodiments disclosed herein. In some embodiments, the fluid transfer module 212 can include the air chamber 208. The air chamber 208 can be in fluid communication with a portion of the source fluid pathway 206 that is inside the fluid transfer module 212. As illustrated, the air chamber can be configured to be sealed off or isolated from ambient air when fluid is flowing through or present in the transfer module 212. The air chamber 208 can be located between the source interface and the intermediate container 204, or between the source interface and the intermediate interface. The fluid transfer module can include an interaction portion 232 that is configured to permit the air sensor to detect air at a sensing location, which can be in the source fluid pathway 206. In some embodiments, the interaction portion 232 can be substantially transparent to the light (e.g., visible light, near infrared (NIR), infrared (IR) light, etc.) used by the air sensor 210. Some small amount of light can be absorbed, reflected, etc. as the light of the air sensor passes through the interaction portion 232 of the fluid transfer module 212, but the interaction portion 232 can be substantially transparent such that sufficient light is transmitted to enable the air sensor 210 to reliably distinguish between fluid and air. The interaction portion 232 can be substantially flat, which can facilitate the passage of light through the sensing location substantially unchanged (e.g., without being significantly refracted, scattered, or otherwise diverted from the intended light path). In some embodiments, the interaction portion 232 can include some small amount of surface imperfections or irregularities that deviate from being perfectly flat, but the interaction portion 232 can be substantially flat to enable the air sensor 210 to reliably distinguish between fluid and air.

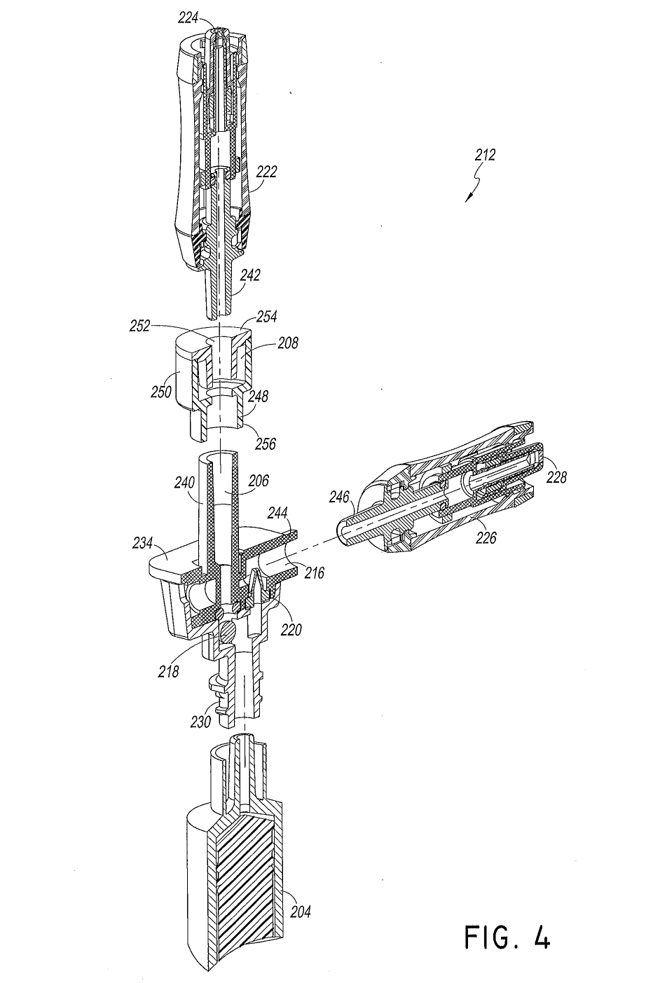

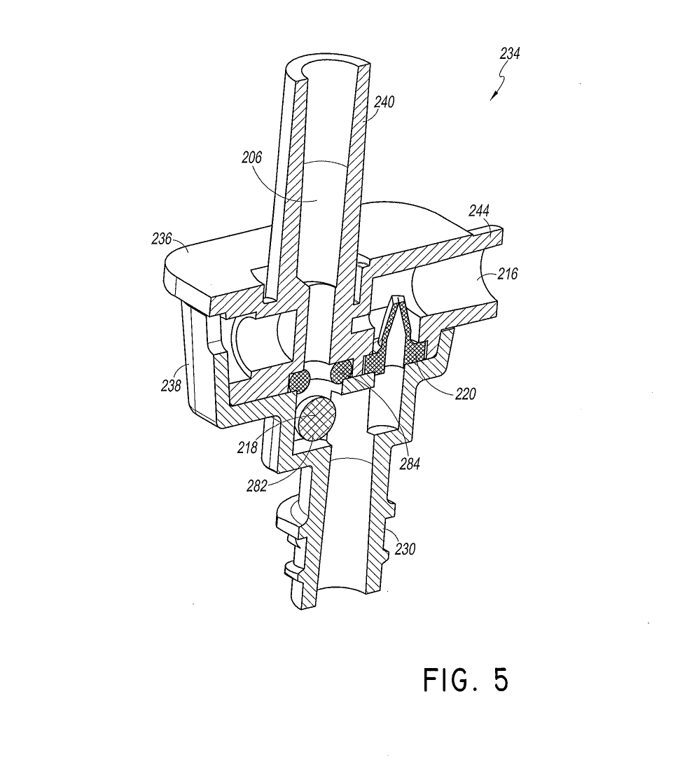

In some embodiments, the fluid transfer module 212 can include a main body 234. FIG. 5 is a perspective, cross-sectional view of an example embodiment of a main body 234. FIG. 6 is an exploded, perspective, cross-sectional view of the main body 234 of FIG. 5. The main body 234 can have any feature of the other embodiments disclosed herein and/or the embodiments described in the '832 patent and/or the '911 Publication. The fluid pathways 206 and/or 216 can extend through the main body 234. The main body 234 can include a first portion 236 (e.g., an upper portion) and a second portion 238 (e.g., a lower portion), which can fit together (e.g., using a snap fit, interference fit, clamp, sonic welding, adhesive, or other suitable attachment mechanism). Portions of the first portion 236 and the second portion 238 can be spaced apart from each other to form portions of the fluid pathways 206 and/or 216 therebetween. The source check valve 218 and/or the destination check valve 220 can be disposed between the first portion 236 and the second portion 238.

The source interface 222 and/or the destination interface 226 can be coupled to the main body 234. For example, the main body 234 can include a source attachment portion 240, which can be configured to couple the source interface 222 to the main body 234. The source attachment portion 240 can include, for example, a male or female end. In some embodiments, the source interface 222 can include an attachment portion 242 that is configured to couple the source interface 222 to the main body 234. For example, the attachment portion 242 can include a female or male end. In some embodiments, the source attachment portion 240 of the main body 234 can couple directly to the attachment portion 242 of the source interface 222 (e.g., using a snap fit, interference fit, clamp, sonic welding, adhesive, or other suitable attachment mechanism). In some embodiments, one or more components (e.g., the air chamber 208) can be disposed between the source attachment portion 240 and the attachment portion 242 of the source interface 222. The source interface 222 can include a closable connector (e.g., a closable male connector), such as described in the '832 patent and/or the '911 Publication.

The main body 234 can include a destination attachment portion 244, which can be configured to couple the destination interface 226 to the main body 234. The destination attachment portion 244 can include, for example, a male or female end. In some embodiments, the destination interface 226 can include an attachment portion 246 that is configured to couple the destination interface 226 to the main body 234. For example, the attachment portion 246 can include a female or male end. In some embodiments, the destination attachment portion 244 of the main body 234 can couple directly to the attachment portion 246 of the destination interface 226 (e.g., using a snap fit, interference fit, clamp, sonic welding, adhesive, or other suitable attachment mechanism). In some embodiments, one or more components can be disposed between the destination attachment portion 244 and the attachment portion 246 of the destination interface 226. The destination interface 226 can include a closable connector (e.g., a closable male connector), such as described in the '832 patent and/or the '911 Publication.

The intermediate interface 230 can be integrated with the main body 234, as shown in FIGS. 3 and 4. In some embodiments, the intermediate interface 230 can be a connector that can be coupled to the main body 234 similar to the source interface 222 and/or the destination interface 226. In some embodiments, the intermediate interface 230 can include a valve that can close the aperture of the intermediate interface 230 when the intermediate connector 204 is detached therefrom.

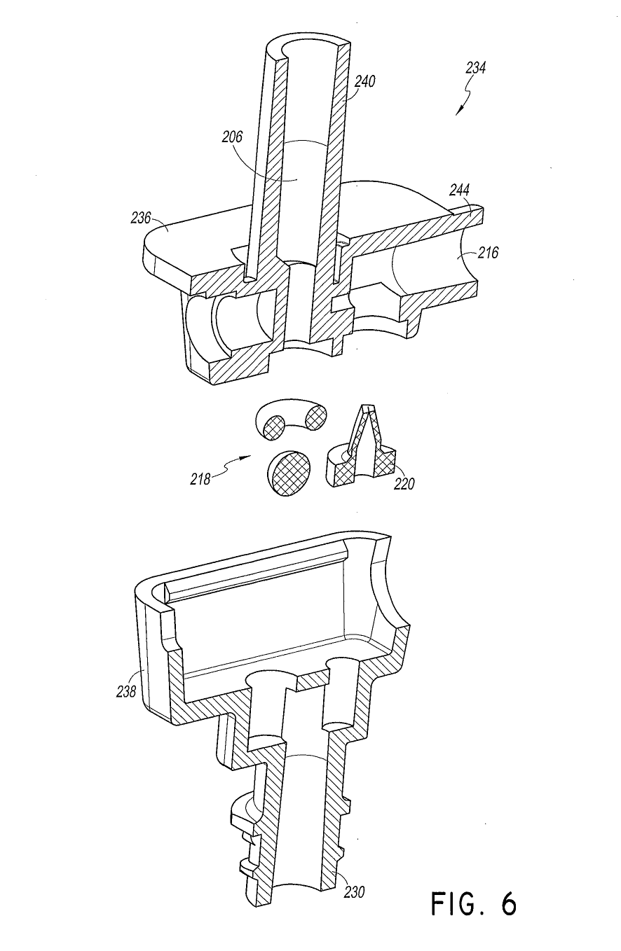

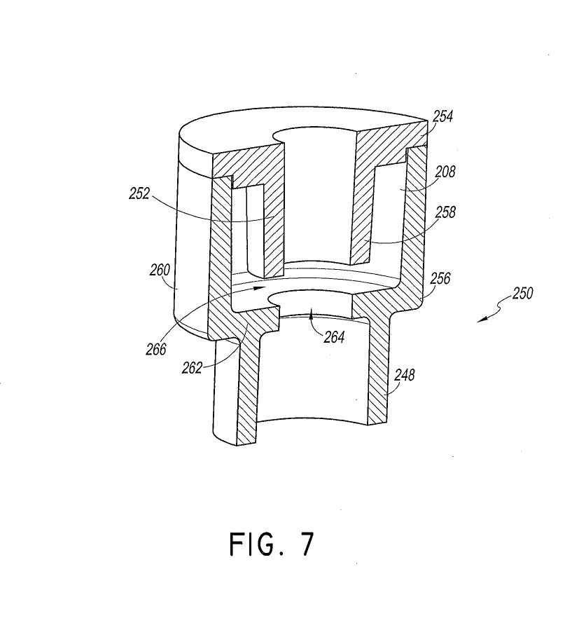

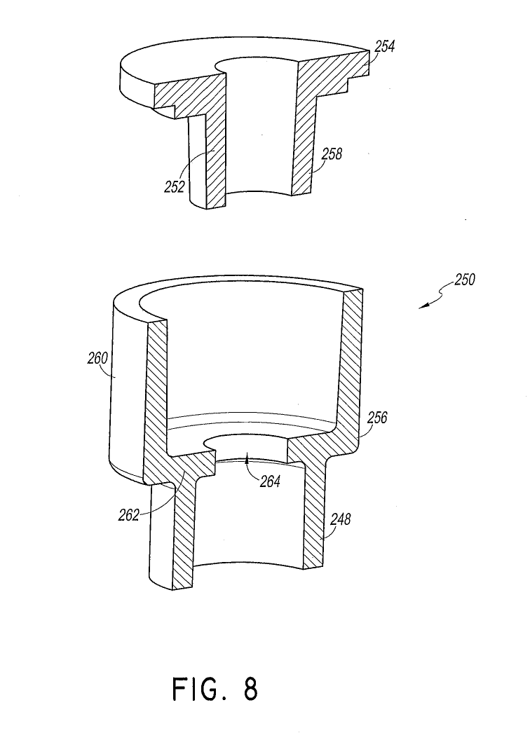

The air chamber 208 can be positioned between the fluid source container 202 and the intermediate container 204. The air chamber 208 can be incorporated as a portion of the fluid transfer module 212. In some embodiments, the air chamber 208 can be disposed between the source interface 222 and the source attachment portion 240. In some embodiments, an air chamber module 250 can include a housing, which can have an internal cavity that forms the air chamber 208. FIG. 7 is a perspective, cross-sectional view of an example embodiment of an air chamber module 250. FIG. 8 is an exploded, perspective, cross-sectional view of the air chamber module of FIG. 7. The air chamber module 250 can include a main body attachment portion 248, which can be configured to couple the air chamber module 250 to the main body 234 (e.g., to the source attachment portion 240). The main body attachment portion 248 can be a female or male end, which can attach to the male or female end of the source attachment portion 240. The air chamber module 250 can be attached to the main body 234 using a snap fit, interference fit, clamp, sonic welding, adhesive, or other suitable attachment mechanism. The air chamber module 250 can include a source attachment portion 252, which can be configured to couple the air chamber module 250 to the source interface 222. For example, the source attachment portion 252 can include a female or male end, which can attach to the male or female end of the attachment portion 224 of the source interface 222. The air chamber module 250 can be attached to the source interface 222 using a snap fit, interference fit, clamp, sonic welding, adhesive, or other suitable attachment mechanism.

The air chamber module 250 can include a first portion 254 (e.g., an upper portion) and a second portion 256 (e.g., a lower portion), which can fit together (e.g., using a snap fit, interference fit, clamp, sonic welding, adhesive, or other suitable attachment mechanism). At least a portion of the first portion 254 can be spaced apart from the second portion 256 to form a gap therebetween, and the gap can include air to provide the air chamber 208. The air chamber module 250 can include a first wall 258 (e.g., an inner wall) and a second wall 260 (e.g., an outer wall) that is spaced apart from the inner wall 258 to form a gap therebetween for the air chamber. The air chamber can have a generally annular shape, in some cases. The first wall 258 can be part of the first portion 154 (e.g., part of the source attachment portion 252, in some embodiments), and the second wall 260 can be part of the second portion 256. A divider 262 can be positioned between the main body attachment portion 248 and the second wall 260. The divider 262 can extend generally transverse to the source fluid pathway 206. The divider 262 can have an aperture 264 that enables fluid to pass through the air chamber module 250. In some embodiments, the second wall 260 can extend in a first direction (e.g., generally upward) from the divider 262, and the main body attachment portion 248 can extend in a second direction (e.g., generally downward) from the divider 262. Those of skill in the art will understand, based on the disclosure herein, that the air chamber 208 and/or the air chamber module 250 can be positioned at various other locations, and that the air chamber module 250 can be modified in various ways from the example embodiments shown in the Figures.

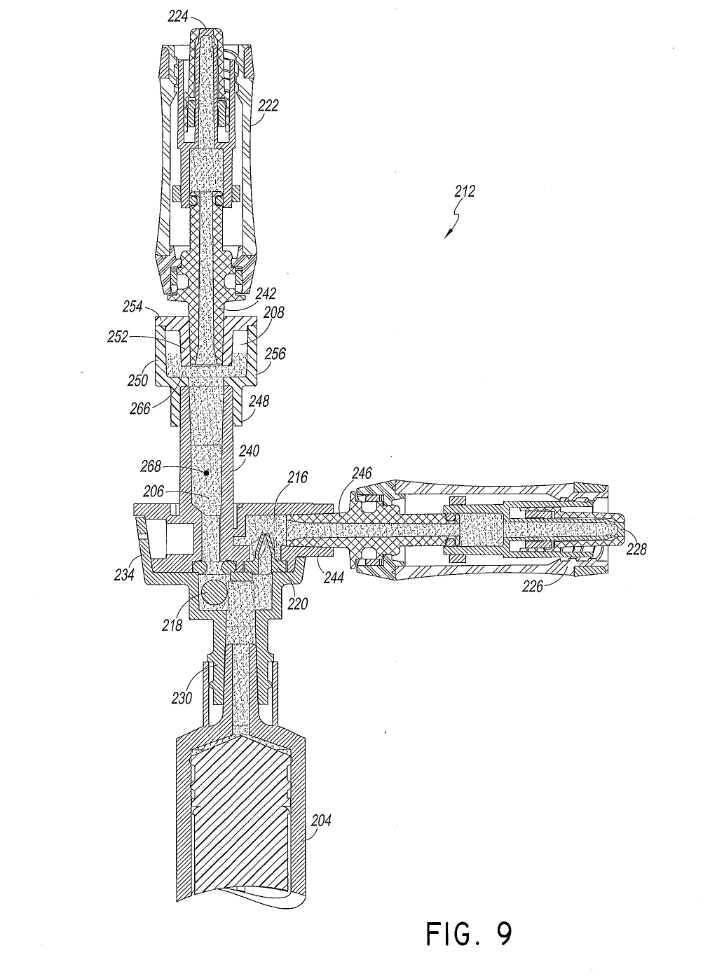

FIG. 9 is a cross-sectional view of the fluid transfer module 212 with fluid in the source fluid pathway 206 and the destination fluid pathway 216. Although FIG. 9 shows the source interface 222 and the destination interface 226 closed and the fluid source container 202 and fluid destination container 214 are omitted for simplicity, it will be understood that during operation the fluid source container 202 can be coupled to the source interface 222 (which can have an open valve 224), and/or the fluid destination container 214 can be coupled to the destination interface 226 (which can have an open valve 228). During operation, the plunger of the syringe 204 can be retracted (e.g., by an actuator on an electronically controlled fluid dispensing system as described in the '832 patent and/or the '911 Publication). Retraction of the plunger can draw fluid from the fluid source container, through the fluid pathway 206, and into the intermediate container 204 (e.g., the syringe). The destination check valve 220 can impede or prevent fluid from flowing from the fluid destination container 214 towards the intermediate container when the plunger is retracted. The source fluid pathway 206 can extend through an adapter (e.g., a vial adapter, not shown in FIG. 9), the source interface 222, the air chamber module 250, and/or the main body 234. The plunger of the syringe 204 can be advanced (e.g., by an actuator on an electronically controlled fluid dispensing system, such as described in the '832 patent and/or the '911 Publication). Advancing the plunger can expel fluid from the intermediate container (e.g., the syringe). The source check valve 218 can impede or prevent the expelled fluid from passing back towards the source fluid container 202. The expelled fluid can pass from the intermediate container 204, through the destination fluid pathway 216, and to the target container 214. The destination fluid pathway 216 can extend through the main body 234, the destination interface 226, and/or an adapter (e.g., tubing attached to an IV bag, not shown in FIG. 9). Precise amounts of fluid can be transferred from the fluid source container 202 to the fluid destination container 214, e.g., by retracting the plunger by a distance that corresponds to the desired amount of fluid and then advancing the plunger to drive the fluid into the fluid destination container 214. Additional details are provided in the '832 patent and/or the '911 Publication.

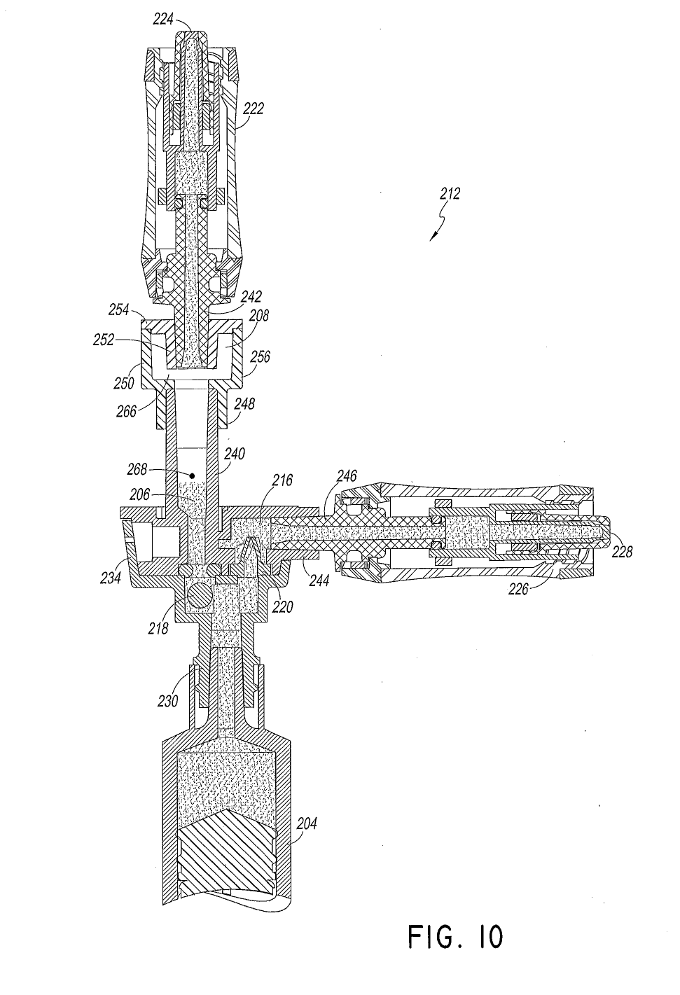

As shown in FIG. 9, in some embodiments, the source fluid pathway can extend through the air chamber module 250. For example, fluid can flow into the air chamber module 250 through the source attachment portion 252 and out through the main body attachment portion 248. The air chamber 208 can be in fluid communication with the source fluid pathway 206. The air chamber 208 can be oriented such that the air is maintained in the air chamber in a generally static mode under normal operating conditions as fluid flows through the source fluid pathway 206. For example, the air chamber 208 can be configured to be at least partially separated from the source fluid pathway 206 in the presence of fluid and/or during fluid flow (e.g., by the first wall 258), and an opening 266 can provide fluid communication between the air chamber 208 and the source fluid pathway 206 (e.g., in a direction that is non-parallel with the first wall 258 and/or non-parallel with the fluid-flow pathway). The opening 266 can be generally annular in shape, in some embodiments. The air chamber 208 can be positioned above the opening 266, such that the air in the air chamber can be positioned above the opening during normal pressure conditions (e.g., during normal operation of the fluid transfer system 200). During operation, in some circumstances, some fluid can pass through the opening 266, and may even enter the lower portion of the air chamber 208. Because the air is less dense than the fluid, the air can rise to the top of the air chamber 208 such that the air does not pass through the opening 266 during normal pressure conditions. As shown in FIG. 9, in some embodiments, during normal operation a continuous stream of fluid can extend through the source fluid pathway 206, through the destination fluid pathway 216, between the source container 202 and the intermediate container 214, between the source container 102 or 202 and the destination container 104 or 204, between the source interface 222 and the intermediate interface 230, and/or between the intermediate interface 222 and the destination interface 226. As can be seen in FIG. 9, the air chamber 208 can be configured such that during normal operation the air is disposed in the air chamber 208 outside a continuous, unbroken path of fluid. The fluid transfer module can be configured, in some embodiments, such that during normal operation fluid flows along the source fluid pathway 206 without passing through the air chamber 208. The air chamber 208 can suspend air outside the source fluid pathway 206 such that the air is in fluid communication with the source fluid pathway 206. As discussed in connection with FIG. 10, under certain circumstances (e.g., when a malfunction or reduced pressure occurs), the air in the air chamber 208 can expand into the source fluid pathway 206 to interrupt the continuous stream of fluid that would normally extend along the source fluid pathway 206.

When a malfunction occurs, e.g., which can impede or prevent fluid from exiting the fluid source container 202, retraction of the plunger can cause reduced pressure (e.g., a partial vacuum) along the fluid pathway. FIG. 10 shows a cross-sectional view of the fluid transfer module 212 with reduced pressure therein (e.g., in the source fluid pathway 206). The reduced pressure can cause the air in the air chamber 208 to expand. In some embodiments, the air can expand through the opening 266 and into the source fluid pathway 206, downstream toward the intermediate interface and/or the intermediate container. The air can expand to reach the sensing location 268. The air sensor 210 (which can be part of the electronically controlled fluid dispensing system, not shown in FIG. 10) can be configured to detect air at the sensing location 268. When the air sensor 210 detects air at the sensing location 268, the air sensor 210 can generate a signal, which can be indicative that a low pressure condition may be present and/or that a malfunction may have occurred. In some embodiments, the electronically controlled fluid dispensing system can stop the fluid transfer (e.g., by stopping retraction of the plunger) in response to the indication from the air sensor 210. In some embodiments, the electronically controlled fluid dispensing system can issue an alarm or issue a notification to a user that a malfunction may have occurred in response to the indication from the air sensor 210.

In some embodiments, the air sensor 210 can be used to detect air as an indication that the fluid source container 202 is empty. Thus the same air sensor 210 can be used to detect an empty fluid source container 202 and to detect a malfunction that produces a low pressure condition. The air sensor 210 can be, positioned to detect air in the source fluid pathway 206. When the fluid source container 202 becomes empty, air can exit the fluid source container 210 and travel through the source fluid pathway 206. When the air reaches the sensing location 268 of the air sensor 210, the air sensor 210 can detect the air. In this situation, the detection of air by the air sensor 210 indicates that the fluid source container 202 has become empty. When a malfunction occurs that results in reduced pressure, the air inside the air chamber 208 can expand into the fluid pathway 206, as discussed herein. When the expanded air reaches the sensing location 268 of the air sensor 210, the air sensor 210 can detect the air. In this situation, the detection of air by the air sensor 210 indicates a reduced pressure inside the fluid pathway 206, which can indicate that a malfunction has occurred. In some embodiments, when the air sensor 210 detects air, a notification can be issued indicating that the source fluid container 202 may be empty (e.g., an need replacement) and/or that a malfunction may have occurred.

The air sensor 210 can be positioned such that the sensing location can be at various different locations. FIG. 11 is a cross-sectional view showing various possible sensing locations 168a-e. The sensing location 268a can be positioned in the source fluid pathway 206 between the air chamber module 250 and the source check valve 218 (e.g., in the source in the source attachment portion 240 of the main body 234 of the fluid transfer module 212. The sensing location 268b can be positioned in the source fluid pathway 206 in the air chamber module 250 (e.g., in the main body attachment portion 248 of the air chamber module 250). In some embodiments, the sensing location 268 can be positioned outside the source fluid pathway 206. For example, the sensing location 268c can be positioned between the air chamber 208 and the source fluid pathway 206, such that the expanding air will reach the sensing location 268c before it enters the source fluid pathway 206. The sensing location 268d can be positioned in the fluid pathway 206 between the source check valve 218 and the intermediate container 204 (e.g., in the intermediate interface 230). In some embodiments, the sensing location 269e can be located inside the intermediate container 204. In some embodiments, multiple sensing locations can be provided in more than one of the foregoing or other locations. Although FIG. 11 shows various different sensing locations 268a-e, it will be understood that a single air sensor 210 can be used for detecting air at one of the shown sensing locations 268a-e. In some embodiments, more than one air sensor 210 can be used. Various other sensing locations can be used, other than the examples shown in the Figures.

In some situations, embodiments that have the sensing location positioned closer to the air chamber 208 can enable the system 200 to detect a malfunction (e.g., a low pressure condition) sooner than embodiments in which the sensing location is positioned further from the air chamber 208. For example, as a low pressure condition develops, the expanding air would reach the upstream sensing location 268c sooner than it would reach the downstream sensing locations 268b or 268a. Thus, by changing the position of the sensing location 268, the sensitivity of the system's ability to detect low pressure can be adjusted. In some cases, positioning the sensing location very close to the air chamber 208 can result in false indications of possible malfunction. For example, the air in the air chamber 208 can move or expand by small amounts during normal operation of the system 200 (e.g., due to acceptable minor changes in pressure or due to movement of the system 200). If the sensing location 268 were positioned very close to the air chamber 208, the acceptable small expansion or movements of the air can cause the air detect 210 to detect the air. The various sensing locations 268a-e shown in FIG. 11 can provide various different balances for the sensitivity of the system's ability to detect low pressure.

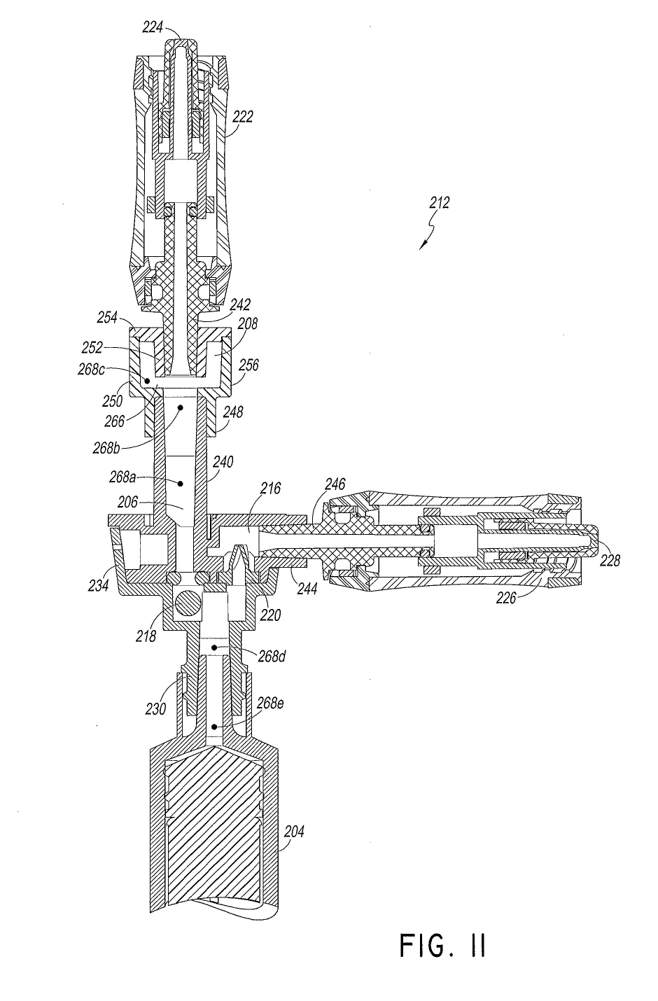

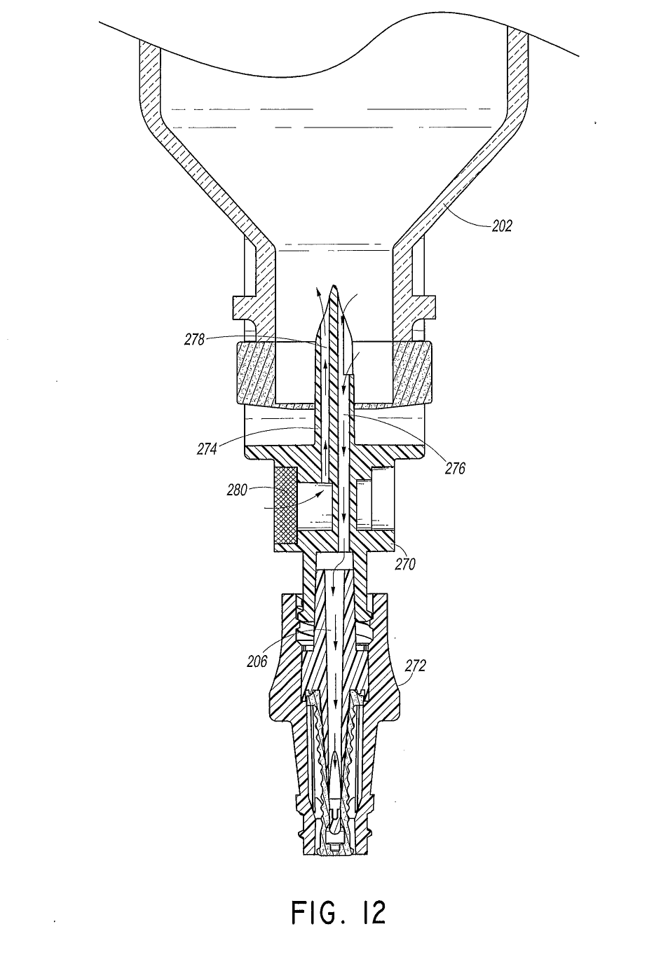

FIG. 12 is a cross-sectional view of a fluid source container 202 and adapter 270, which can have any feature of other embodiments illustrated and/or described in this specification or in the '832 patent and/or the '911 Publication. The fluid source container 202 can be a vial, although other types of containers can be used in some embodiments. The adapter 270 can include a fluid transfer module interface 272, which can be configured to couple the adapter 270 to the fluid transfer module 212. For example, the fluid transfer module interface 272 can be a connector (e.g., a closable female connector) that is configured to removably attach to the source interface 222 (e.g., a closable male connector) of the fluid transfer module 212. The adapter 270 can include a source interface 274, which can be configured to couple to the fluid source container 202. The source interface 274 can include a spike, which can be configured to pierce a septum on the fluid source container 202 (e.g., a vial). The source interface 274 can include a fluid channel 276, which can be configured to permit fluid to exit the fluid source container 202. The fluid channel 276 can form part of the source fluid pathway 206. The source interface 174 can include an air inlet channel 278, which can be configured to permit air to enter the fluid source container 202 (e.g., as fluid exits the fluid source container 202). The volume of the fluid exiting the fluid source container 202 can be replaced by air, thereby impeding or preventing reduced pressure (e.g., a partial vacuum) from developing inside the fluid source container 202.

The adapter 270 can include a barrier 280, which can be generally permeable to air and generally impermeable to fluid. The barrier 280 can allow air to enter the air inlet channel. Under ordinary conditions, the air inlet channel 278 contains air, and the fluid from the fluid source container 202 does not travel through the air inlet channel 278 to the barrier 280. However, in the event that fluid does travel through the air inlet channel 278 to the barrier 280 (e.g., due to improper excessive shaking of the device and/or improper over-pressurization of the fluid source container before connection to the fluid transfer system), the barrier can prevent the fluid from exiting the adapter 270. However, in some situations, if the barrier 280 becomes wet, the air permeability of the barrier 280 can be reduced or eliminated, which can impede or prevent air from entering the air inlet channel 278. If no air, or insufficient air, enters the fluid source container 202 as fluid is drawn out of the fluid source container, a reduced pressure (e.g., a partial vacuum) can form inside the fluid source container 202. The reduced pressure can impede or prevent fluid from exiting the fluid source container 202 (e.g., as the plunger of the syringe 204 is retracted). The reduced pressure can spread to areas that are in fluid communication with the fluid source container 202 (e.g., the air chamber 208). The air in the air chamber 208 can expand to compensate for the reduced pressure, and the air sensor 210 can detect the expanded air, as discussed herein. In some embodiments, the air in the air chamber 208 can have a first volume during normal operating pressure (e.g., as shown in FIG. 9). When the pressure in the source fluid pathway 206 drops below a threshold value (e.g., due to an impediment in the barrier 280), the air can expand to a second volume that is larger than the first volume and that is large enough to position air at the sensing location 268). Those of skill in the art will understand, based on the disclosure herein, that various other types of malfunctions can occur (e.g., a collapsed tubing, a compromised connector, a malfunctioning check valve, etc.), which can result in reduced pressure, and that systems and methods similar to those discussed herein can be used to detect the malfunctions.

With reference again to FIGS. 5 and 6, various different check valve configurations can be used for the source check valve 218 and/or the destination check valve 220. For example, a duck bill check valve can be used (e.g., see the destination check valve 220 shown in FIGS. 5 and 6). Various other check valve configurations disclosed in the '832 patent and/or the '911 Publication can be used. As shown in FIGS. 5 and 6, the source check valve 218 can include a stopper 282. The stopper 282 can be generally spherical in shape (e.g., a ball check valve), although other shapes can also be used for the stopper 282. The stopper 282 can be movable between a closed position and an open position. The “open” position should be understood according to its ordinary meaning in this field, and includes a position that permits sufficient fluid flow to perform the clinical function(s) for which the product is intended. The “closed” position should be understood according to its ordinary meaning in this field, and includes a position that completely obstructs fluid flow within normal ranges of fluid pressure in a particular intended application for the product in which it is intended to be used, or that substantially entirely obstructs fluid flow to the degree necessary to avoid clinically significant functional disadvantages. FIG. 5 shows the stopper 282 in the open position. The check valve 218 can include a sealing element 284, and the stopper 282 can contact or abut against the sealing element 284 when the stopper 282 is in the closed position. The sealing element 284 can be configured to seal against the stopper 282 when the stopper 282 is in the closed position. For example, the sealing element 284 can be formed of a resilient material (e.g., rubber, silicone, latex, etc.) and the stopper 282 can be formed of a rigid or semi-rigid material (e.g., a hard plastic or metal). The sealing element 284 can deform when the stopper 282 abuts against the sealing element 284 to form a seal. In some embodiments, the sealing element 284 can be a rigid or semi-rigid material and the stopper 282 can be a resilient material, or both the sealing element 284 and the stopper 282 can be formed of resilient materials. In some embodiments, the sealing element 284 can be one or more interior walls of the fluid transfer module 212, and the stopper 282 can be configured to seal against the one or more interior walls of the fluid transfer module 212. In some embodiments, the sealing element 284 can be generally circular. As illustrated, the sealing element 284 can comprise an O-ring. In some embodiments, an internal fluid-flow pathway through the sealing element 284 can comprise an aperture with a diameter or cross-sectional width that is less than the diameter or cross-sectional width of the stopper 282.

When fluid flows in a first direction (e.g., from the fluid source container 202 toward the intermediate container 204), the stopper 282 can move to the open position that is spaced apart from the sealing element 284 to allow fluid to flow through the check valve 218 (e.g., when the plunger is retracted on the syringe 204). The check valve 218 can impede or prevent fluid from passing through the check valve 218 in a second direction (e.g., from the intermediate container 204 toward the fluid source container 202). When fluid is urged to flow in the second direction (e.g., when the plunger on the syringe 204 is advanced), the fluid can push the stopper 282 in the direction of the sealing element, and press the stopper 282 tightly against the sealing element 284, and fluid can be prevented or impeded from passing through the check valve 218 in the second direction. In some embodiments, the stopper 282 can be biased toward the closed position. In some embodiments, the stopper can be less dense than the fluid, such that the stopper 282 tends to float up into contact with the sealing element 284 in the presence of fluid. In some embodiments, as illustrated in FIG. 5, the stopper 282 can be free floating (e.g., not directly coupled or secured to surrounding elements) within a chamber. The chamber can be sized such that the stopper 282 can move between the closed position and the open position, but the stopper 282 cannot exit the chamber. For example, a diameter or cross-sectional width of the stopper 282 can be greater than a diameter or cross-sectional width of a downstream fluid-flow aperture in the region where the stopper 282 is configured to be located. In some embodiments, to enable insertion of the stopper 282 during manufacturing, a diameter or cross-sectional width of the stopper 282 can be less than or about equal to a diameter or cross-sectional width of an upstream fluid-flow entrance aperture in the region where the stopper 282 is configured to be located.

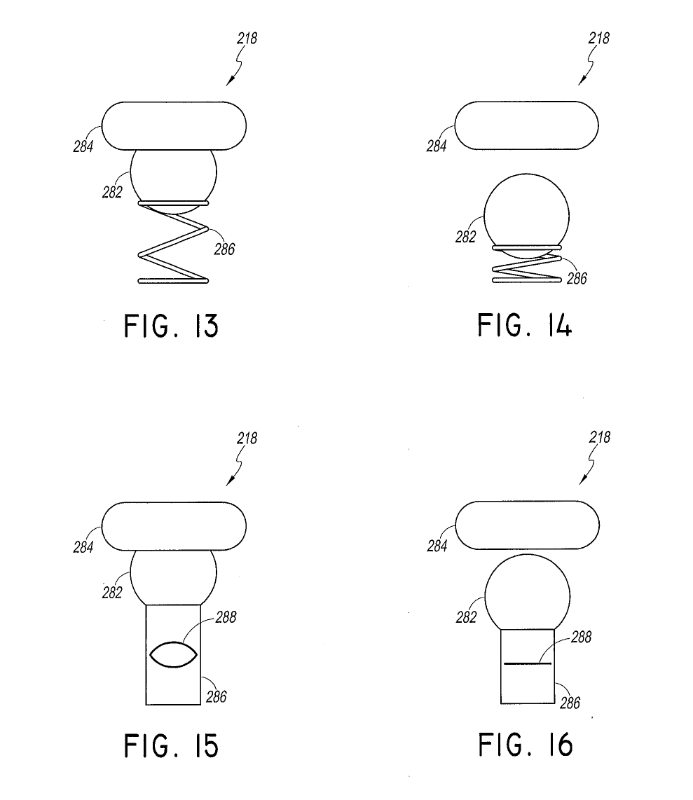

With reference to FIGS. 13-18, in some embodiments, a biasing element 286 can bias the stopper 282 toward the closed position. The biasing element 286 can be a spring. For example, as shown in FIGS. 13 and 14, the biasing element 286 can be a helical coil spring. FIG. 13 shows a side view of an example embodiment of the check valve 218 in the closed position. In the closed position, the biasing element 286 (e.g., coil spring) presses the stopper 282 (e.g., ball) against the sealing element 284 to prevent or impede fluid from flowing through the check valve 218. FIG. 14 shows the check valve 218 of FIG. 13 in the open position. In the open position, the coil spring 286 can be compressed, and the stopper 282 can be spaced apart from the sealing element 284 such that fluid can pass through the check valve 218.

FIG. 15 shows a side view of another example embodiment of a check valve 218 in the closed position. FIG. 16 is a side view of the check valve 218 of FIG. 15 in the open position. The biasing element 286 can be a resilient body that can deform when the stopper 282 is pushed to the open position (FIG. 16). The resilient body can be biased to return to its undeformed shape, which can apply a force that presses the stopper 282 towards the sealing element 284. In some embodiments, the resilient body of the biasing element 286 can have at least one cavity 288. When the check valve 218 is in the closed position, the cavity 288 can be in an at least partially open configuration (as shown in FIG. 15). When the check valve 218 is in the open position, the cavity 288 can be in an at least partially collapsed position (as shown in FIG. 16). The collapse of the at least one cavity 288 can facilitate the deformation of the resilient body from the closed position to the open position.

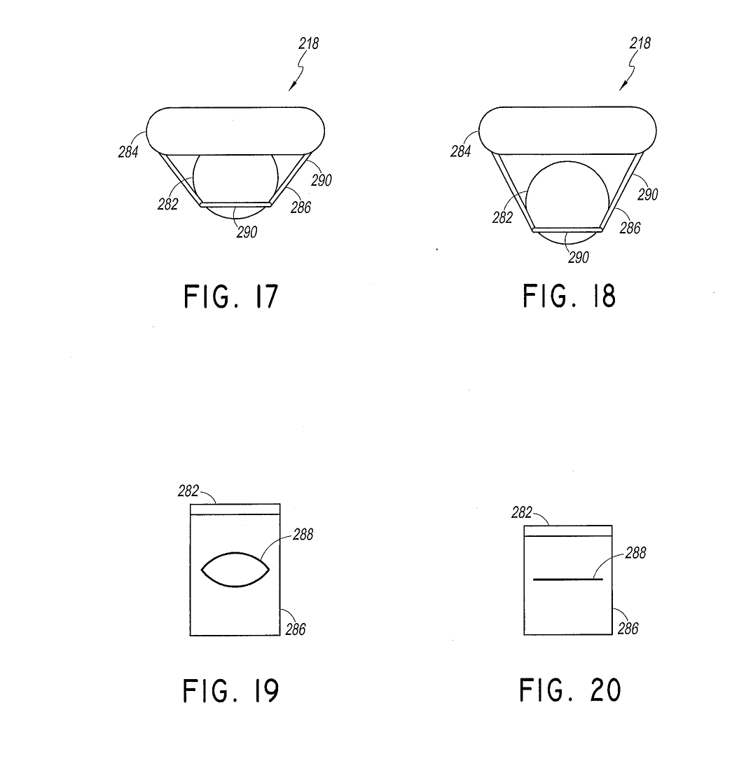

FIG. 17 shows a side view of another example embodiment of a check valve 218 in the closed position. FIG. 18 is a side view of the check valve 218 of FIG. 17 in the open position. The biasing element 286 can include one or more flexible tethers 290, which can pull the stopper 282 against the sealing element 284. When the check valve 218 is in the closed position, the one or more tethers 290 can have a first length (as shown in FIG. 17). When the check valve 218 is in the open position, the one or more tethers 290 can stretch to have a second length that is longer than the first length (as shown in FIG. 18). The one or more tethers 290 can be coupled to the stopper 282 (e.g., via a coupling element 292, which can be an annular element that has a diameter that can be smaller than the diameter of the stopper 282). In some embodiments, the one or more tethers 290 can be coupled to the sealing element 284 (e.g., the one or more tethers 290 can be integrally formed with the sealing element 284). In some embodiments, the one or more tethers 290 can be coupled to the interior walls that surround the check valve 218.

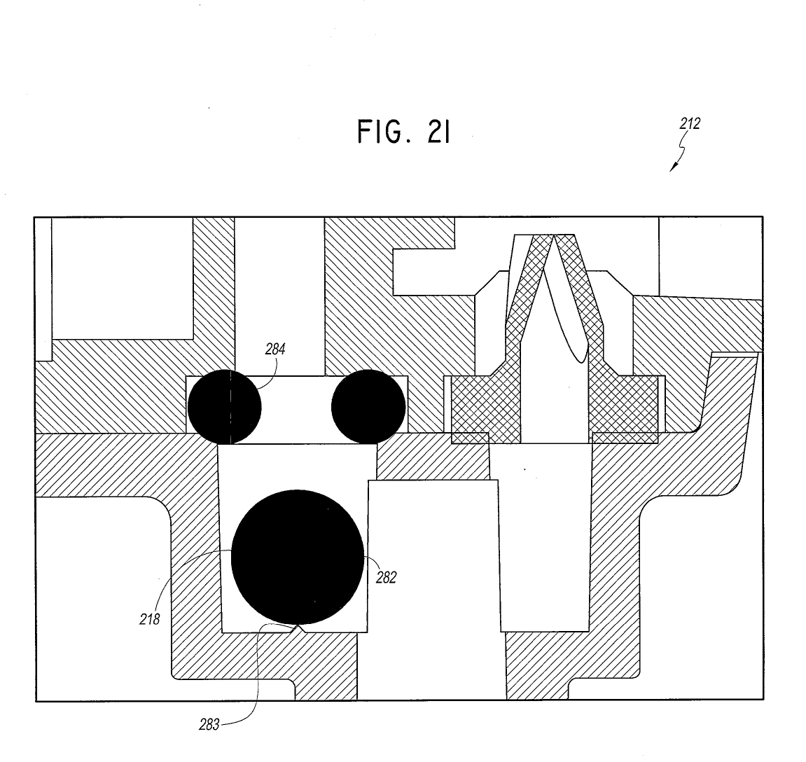

FIG. 19 is a side view of another example embodiment of a check valve 218 in the closed position. FIG. 20 is a side view of the check valve 218 of FIG. 20 in the open position. In some embodiments, the biasing element 286 and stopper 282 can be incorporated into a single element. For example, the check valve 218 can include a stopper portion 282 (e.g., an upper portion), which can be configured to seal against a sealing element 284 (e.g., against a resilient member or against an interior wall of the fluid transfer module 212). The check valve 218 can include a biasing element 286, which can be a resilient body that can be configured to bias the stopper portion 282 towards the closed position, e.g., in a manner similar to the embodiments discussed in connection with FIGS. 15 and 16. The biasing element 286 can include at least one cavity 288. When the check valve 218 is in the closed position, the cavity 288 can be in an at least partially open configuration (as shown in FIG. 19). When the check valve 218 is in the open position, the cavity 288 can be in an at least partially collapsed position (as shown in FIG. 20). The collapse of the at least one cavity 288 can facilitate the deformation of the biasing element 286 from the closed position to the open position. In some embodiments, the stopper portion 282 can be integrally formed with the biasing element 286. In some embodiments, the stopper portion 282 can be formed separately from the biasing element 186 and can be coupled to the biasing element 286 (e.g., by an adhesive or other suitable attachment mechanism).

In FIGS. 13-20, the check valves 218 are shown in isolation and the surrounding components (e.g., the fluid transfer module 212) are omitted from view for simplicity. FIGS. 12-20 show example embodiments of the source check valve 218. The destination check valve 220 can include features that are similar to, or the same as, the features shown (e.g., in FIGS. 13-20) and discussed in connection with the source check valve 218.

FIG. 21 is a cross-sectional view of an example embodiment of a fluid transfer module 212 that includes at least one check valve. The check valve 218 can include a stopper portion 282 that is movable between a closed position and an open position, as described herein. FIG. 21 shows the stopper portion 282 in an open position. The fluid transfer module 212 can include a protrusion 283, which can be configured to contact the stopper portion 282 when the stopper portion 282 is in the open position (e.g., in a fully open position). The protrusion 283 can be configured to facilitate the transition of the stopper portion 282 from the open position to the closed position (e.g., when flow of fluid or a fluid pressure urges the stopper portion toward the closed position). For example, the protrusion 283 can impede or prevent the stopper portion 282 from sticking to an internal wall of the fluid transfer module 212. The protrusion 283 can extend (e.g., upward) from the internal wall so that when the stopper portion 282 moves towards the internal wall, the stopper portion 282 contacts or abuts the protrusion 283. The protrusion 283 can be configured to space the stopper portion 282 away from the interior wall when the stopper portion 282 is in the open position. In some embodiments, the gap between the stopper portion 282 in the open position and the interior wall adjacent or near the protrusion 283 can facilitate the transition of the stopper portion 282 to the closed position. For example, when a fluid flow or fluid pressure is applied, fluid can enter the gap between the stopper portion 282 and the interior wall and can push the stopper portion 282 away from the interior wall and towards the closed position. In some embodiments, the contact area between the stopper portion 283 and the protrusion can be less than the contact area that would result between the stopper portion 282 and the internal wall if the protrusion 283 were omitted. The protrusion 283 can include a tip portion that can be pointed or rounded to reduce contact area between the stopper portion 282 and the protrusion. The tip portion can also have a flat shape, and various other shapes can be used. In some embodiments, the fluid transfer module 212 can include a plurality of protrusions 283, which can be configured to facilitate movement of the stopper portion 282. In some embodiments, the protrusion 283 can be an elongate ridge.

Example embodiments have been described in connection with the accompanying drawings. The foregoing example embodiments have been described at a level of detail to allow one of ordinary skill in the art to make and use the devices, systems, methods, etc. described herein. Those of skill in the art will understand, based on the disclosure herein, that a wide variety of variations are possible. Components, elements, and/or steps may be altered, added, removed, or rearranged. Additionally, processing steps may be added, removed, or reordered. While certain embodiments have been explicitly described, other embodiments will also be apparent to those of ordinary skill in the art based on this disclosure.

Some aspects of the systems and methods described herein can advantageously be implemented using, for example, computer software, hardware, firmware, or any combination of software, hardware, and firmware. Software can comprise computer executable code for performing the functions described herein. In some embodiments, computer-executable code is executed by one or more general purpose computers. However, a skilled artisan will appreciate, in light of this disclosure, that any module that can be implemented using software to be executed on a general purpose computer can also be implemented using hardware, software, firmware, or combinations thereof. For example, such a module can be implemented completely in hardware using a combination of integrated circuits. Alternatively or additionally, such a module can be implemented completely or partially using specialized computers designed to perform the particular functions described herein rather than by general purpose computers.

While certain embodiments have been explicitly described, other embodiments will become apparent to those of ordinary skill in the art based on this disclosure. Therefore, the scope of the invention is intended to be defined by reference to the claims as ultimately published in one or more publications or issued in one or more patents and not simply with regard to the explicitly described embodiments.

The following is claimed:

1. A method of transferring fluid from a fluid source container to a syringe, the method comprising:
retracting a plunger on a syringe to draw a first volume of fluid from a fluid source container, through a source fluid pathway, and into the syringe, wherein an air chamber is in fluid communication with the source fluid pathway between the fluid source container and the syringe;
retracting the plunger on the syringe to draw a second volume of fluid from the source fluid pathway into the syringe, wherein fluid is impeded from exiting the source container such that air in the air chamber expands to a sensing location in the fluid pathway between the source container and the syringe; and
identifying the air at the sensing location in the source fluid pathway between the source container and the syringe with an air sensor; and
in response to identifying the air at the sensing location in the fluid pathway, stopping the retracting of the plunger on the syringe.

2. The method of claim 1, further comprising:
in response to identifying the air at the sensing location in the fluid pathway, providing an indication that a malfunction may have occurred.

3. The method of claim 1, further comprising:
in response to identifying the air at the sensing location in the fluid pathway, providing an indication that the fluid source container may be empty.

4. The method of claim 1, wherein the first volume of fluid passes through a source check valve in the source fluid pathway between the fluid source container and the syringe, wherein the source check valve is configured to impede fluid from passing through the source check valve towards the fluid source container.

5. The method of claim 1, further comprising advancing the plunger on the syringe to drive fluid from the syringe, through a destination fluid pathway, and towards a fluid destination container.

6. The method of claim 5, wherein the fluid passes through a destination check valve in the fluid destination pathway between the syringe and the fluid destination container, wherein the destination check valve is configured to impede fluid from passing through the destination check valve towards the syringe.
